(19) United States
(12) Patent Application Publication
(10) Pub. No.: US 2026/0130633 A1
(43) Pub. Date: May 14, 2026

(54) SYSTEM AND METHOD FOR PREDICTING DISEASE RISK IN FEMALE

(71) Applicants: TAIPEI MEDICAL UNIVERSITY, Taipei City (TW); FlouriSHE Limited Company, Taipei City (TW)

(72) Inventors: Chih-Sheng HSU, New Taipei City (TW); Phung-Anh NGUYEN, New Taipei City (TW); Chih-Wei HUANG, New Taipei City (TW); Jui-Pin CHANG, Taipei City (TW)

(73) Assignees: TAIPEI MEDICAL UNIVERSITY, Taipei City (TW); FlouriSHE Limited Company, Taipei City (TW)

(21) Appl. No.: 19/389,768

(22) Filed: Nov. 14, 2025

Related U.S. Application Data
(60) Provisional application No. 63/720,728, filed on Nov. 14, 2024.

(51) Int. Cl. A61B 5/00; A61B 5/0205; G01N 33/574

(52) U.S. Cl. CPC A61B 5/7275; A61B 5/0205; A61B 5/4306; A61B 5/4509; A61B 5/4836; A61B 5/7267; A61B 5/7435; G01N 33/575

(57) ABSTRACT

Provided are a system and method for predicting a disease risk of a female subject in need thereof, including an input module, a sub-outcome analysis module, an outcome analysis module, an outcome display module, and a sub-outcome display module. The input module is configured to input feature data of the female subject. The sub-outcome analysis module is configured to generate a sub-outcome corresponding to a risk of a comorbidity of a disease based on to the feature data. The outcome analysis module is configured to generate an outcome corresponding to a risk of the disease based on a comprehensive assessment of the sub-outcome. The outcome display module is configured to present the outcome on a first operation interface. The sub-outcome display module is configured to present the sub-outcome on a second operation interface in response to an input selection made via the first operation interface.

### BACKGROUND

### Technical Field

The present disclosure relates to systems and methods for predicting disease risks in females.

### Description of Related Art

A comprehensive and informative assessment and prediction of disease risks is important for an individual to receive proper treatment and have a quality life. Before the advent of information technology, this kind of assessment and prediction is conducted and greatly depends on the experience and expertise of a physician or a clinician. However, manual assessment and prediction by a physician or a clinician may be affected by subjective point-of-view, time restriction, and processing ability of the physician or the clinician with respect to a large amount of information.

There have been attempts to utilize artificial intelligence techniques in the medical field to assist in analyzing disease risks in an individual. However, current attempts all fail to comprehensively consider physiological data, individual differences, genetic information, and/or living conditions for a personalized assessment and prediction of disease risks.

Therefore, there is an unmet need in the art to develop a system and a method for predicting a disease risk in a female that can be implemented effectively and simultaneously address the above problems.

### SUMMARY

The present disclosure provides a system for predicting a disease risk of a female subject in need thereof, the system comprising an input module, a sub-outcome analysis module, an outcome analysis module, an outcome display module, and a sub-outcome display module. In at least one embodiment of the present disclosure, the input module is configured to input feature data of the female subject in need thereof. In at least one embodiment of the present disclosure, the sub-outcome analysis module is coupled with the input module and is configured to generate a sub-outcome corresponding to a risk of a comorbidity of a disease according to the feature data. In at least one embodiment of the present disclosure, the outcome analysis module is coupled with the sub-outcome analysis module and is configured to generate an outcome corresponding to a risk of the disease according to a comprehensive assessment of the sub-outcome. In at least one embodiment of the present disclosure, the outcome display module is coupled with the outcome analysis module and is configured to present the outcome on a first operation interface of the outcome display module. In at least one embodiment of the present disclosure, the sub-outcome display module is coupled with the sub-outcome analysis module and the outcome display module and is configured to present the sub-outcome on a second operation interface of the sub-outcome display module in response to an input selection made via the first operation interface.

The present disclosure further provides a method for predicting a disease risk of a female subject in need thereof, the method comprising inputting feature data of the female subject in need thereof into an input module, generating a sub-outcome corresponding to a risk of a comorbidity of a disease according to the feature data by a sub-outcome analysis module, generating an outcome corresponding to a risk of the disease according to a comprehensive assessment of the sub-outcome by an outcome analysis module, displaying the outcome on a first operation interface of the outcome display module, and displaying the sub-outcome on a second operation interface of the sub-outcome analysis module in response to an input selection made via the first operation interface of the outcome display module.

In at least one embodiment of the present disclosure, the feature data is at least one of a demographic data, a cancer status, presence of a comorbidity, treatment received, medication received, laboratory test data, and any combination thereof.

In at least one embodiment of the present disclosure, the demographic data is age, body mass index, smoking status, drinking status, or betel nut chewing status.

In at least one embodiment of the present disclosure, the cancer status is tumor size, cancer stage, expression of human epidermal growth factor receptor 2, expression of estrogen receptor, expression of progesterone receptor, or a Ki-67 proliferation index.

In at least one embodiment of the present disclosure, the presence of a comorbidity is the presence of hypertension, hyperlipidemia, renal disease, lung disease, diabetes, cerebrovascular disease, liver disease, congestive heart failure, dementia, rheumatic disease, or peptic ulcer disease.

In at least one embodiment of the present disclosure, the presence of a comorbidity is a Charlson comorbidity index score.

In at least one embodiment of the present disclosure, the treatment is a surgery or a radiotherapy.

In at least one embodiment of the present disclosure, the medication is biguanide drugs, 3-hydroxy-3-methylglutaryl-coenzyme A reductase inhibitors, platelet aggregation inhibitors excluding heparin, beta-blocking agents, dihydropyridine derivatives, angiotensin II receptor blockers, benzodiazepine derivatives, dipeptidyl peptidase-4 inhibitors, sulfonylureas, or cyclooxygenase-2 inhibitors.

In at least one embodiment of the present disclosure, the laboratory test data is an albumin level, a creatinine level, a bilirubin level, an aspartate aminotransferase (AST) level, an alanine transaminase (ALT) level, a fasting glucose level, a white blood cell count, a red blood cell count, or a platelet count.

In at least one embodiment of the present disclosure, the disease is a bone mass density-related disease, a metabolic syndrome, or a cancer.

In at least one embodiment of the present disclosure, the comorbidity is a bone mass density condition, osteoporosis, hypertension, hyperlipidemia, hyperglycemia, a drug response related to ongoing hormone replacement therapy, ovarian cancer, endometrial cancer, cervical cancer, colorectal cancer, or breast cancer.

In at least one embodiment of the present disclosure, the disease is a bone mass density-related disease, and the comorbidity is a bone mass density condition, osteoporosis, or a drug response related to ongoing hormone replacement therapy. In at least one embodiment of the present disclosure, the bone mass density condition is a normal condition, a predicted deterioration, or a predicted improvement.

In at least one embodiment of the present disclosure, the disease is a metabolic syndrome, and the comorbidity is hypertension, hyperlipidemia, hyperglycemia, or a drug response related to ongoing hormone replacement therapy.

In at least one embodiment of the present disclosure, the disease is a cancer, and the comorbidity is ovarian cancer, endometrial cancer, cervical cancer, colorectal cancer, breast cancer, or a drug response related to ongoing hormone replacement therapy.

In at least one embodiment of the present disclosure, the sub-outcome analysis module is configured to generate, by an artificial intelligence model, the sub-outcome according to the feature data.

In at least one embodiment of the present disclosure, the outcome analysis module is configured to generate, by an artificial intelligence model, the outcome according to a comprehensive assessment of the sub-outcome.

In at least one embodiment of the present disclosure, the artificial intelligence model is configured to perform an algorithm including a logistic regression, a linear discriminant analysis, a light gradient boosting machine, a gradient boosting machine, an extreme gradient boosting machine, an adaptive boosting machine, a support vector classifier, a voting ensemble, a random forest, an artificial neural network, or any combination thereof.

### BRIEF DESCRIPTION OF DRAWINGS

The present disclosure can be more fully understood by reading the following detailed descriptions of the embodiments, with reference made to the accompanying drawings.

FIG. 1 shows a schematic diagram of a system of the present disclosure for predicting a disease risk of a female subject according to at least one embodiment of the present disclosure.

FIG. 2 shows a schematic diagram of application scenarios for the system of the present disclosure for predicting a disease risk of a female subject according to at least one embodiment of the present disclosure.

FIG. 3 shows a flow chart of a method of the present disclosure for predicting a disease risk of a female subject according to at least one embodiment of the present disclosure.

FIG. 4 shows a schematic diagram of a radar chart on a first operation interface displayed by an outcome display module according to at least one embodiment of the present disclosure.

FIG. 5 shows a schematic diagram of a two-dimensional scatter chart on a second operation interface displayed by a sub-outcome display module according to at least one embodiment of the present disclosure.

FIG. 6 shows a flow chart of a study for building a prediction model of 5-year survival of breast cancer patients according to at least one embodiment of the present disclosure.

FIG. 7 shows the receiver operator characteristic (ROC) curve of different machine learning models of the first external validation according to at least one embodiment of the present disclosure.

FIG. 8 shows the ROC curve of the second external validation according to at least one embodiment of the present disclosure.

### DETAILED DESCRIPTION

The following descriptions of the embodiments illustrate implementations of the present disclosure, and those skilled in the art of the present disclosure can readily understand the advantages and effects of the present disclosure and/or apply the present disclosure to other embodiments in accordance with the contents herein. Therefore, any factors described in the present disclosure may be combined with any other factors disclosed in embodiments of the present disclosure.

As used herein, when describing an object “comprises,” “includes,” “contains,” or “has” an element, structure, region, part, apparatus, device, system, step, connection, module, unit, etc., unless otherwise specified, it may additionally encompass other elements, structures, regions, parts, apparatus, devices, systems, steps, connections, modules, units, etc., and should not exclude others. Further, unless otherwise specified, wordings in singular forms such as “a,” “an,” and “the” also pertain to plural forms, and wordings such as “or” and “and/or” may be used interchangeably.

As used herein, the phrase “at least one,” in reference to a list of one or more elements, should be understood to mean at least one element selected from any one or more of the elements in the list of elements but not necessarily including at least one of each and every element listed within the list of elements and not excluding any combinations of elements in the list of elements. This definition may also allow elements to be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The terms “first,” “second,” etc. used herein are simply used to describe or distinguish elements such as components, structures, regions, parts, apparatus, devices, systems, steps, connections, modules, units, etc., rather than used to limit the scope of implementation of the present disclosure or to limit the temporal or spatial order of the elements.

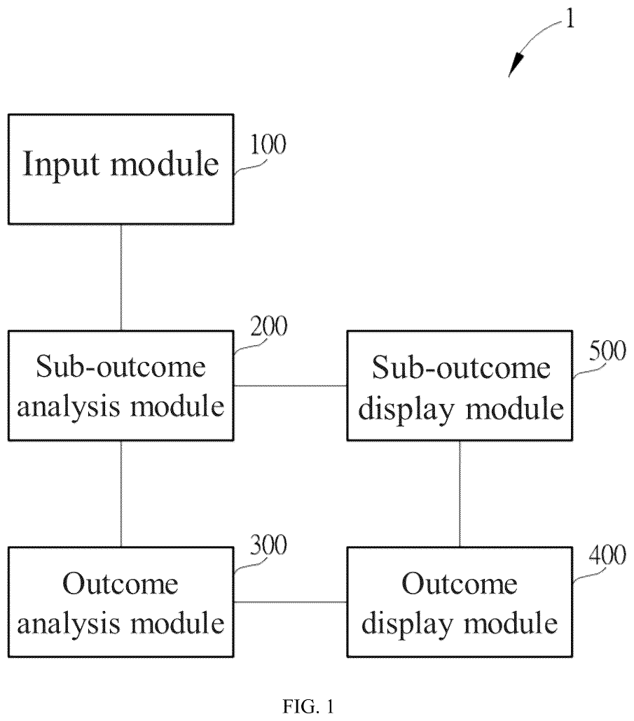

In at least one embodiment, FIG. 1 shows a schematic diagram of a system 1 of the present disclosure for predicting a disease risk of a female subject. In at least one embodiment of the present disclosure, the system 1 includes an input module 100, a sub-outcome analysis module 200, an outcome analysis module 300, an outcome display module 400, and a sub-outcome display module 500. In some embodiments of the present disclosure, the above elements of the system 1 may be coupled with each other via any appropriate wired or wireless connection. Moreover, in at least one embodiment of the present disclosure, the system 1 may be embedded in a Software-as-a-Service (SaaS) environment, an examination apparatus, and/or an examination environment accessible for an arbitrary medical facility. In some embodiments of the present disclosure, the system 1 may be accessed by a subject in need thereof or an operator at a medical facility conducting an examination on the subject. In some embodiments of the present disclosure, the subject may be a female. For example, in one scenario, the system 1 may be used as an auxiliary tool for general patient triage in a general practice by providing personal prevention and treatment strategies based on each subject's risk results before referring them to specialist clinics.

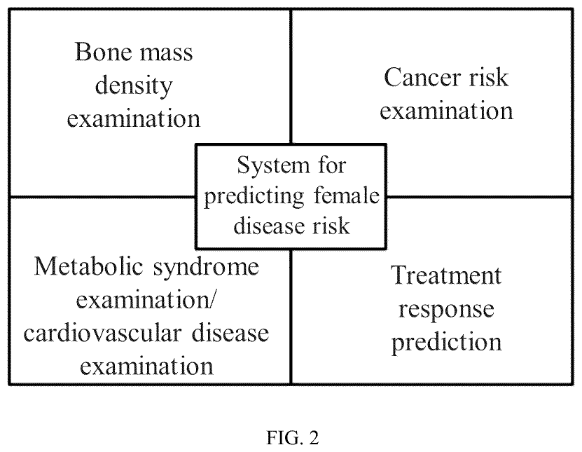

Further, as shown in FIG. 2, in at least one embodiment of the present disclosure, the system 1 for predicting a disease risk of a female may be applied in the field of, e.g., bone mass density examination, metabolic syndrome examination, cardiovascular disease examination, cancer risk examination, or treatment response prediction for the subject. In some embodiments, the above clinical uses for the system 1 of the present disclosure may be conducted individually or simultaneously on a subject (e.g., a female) according to examination needs.

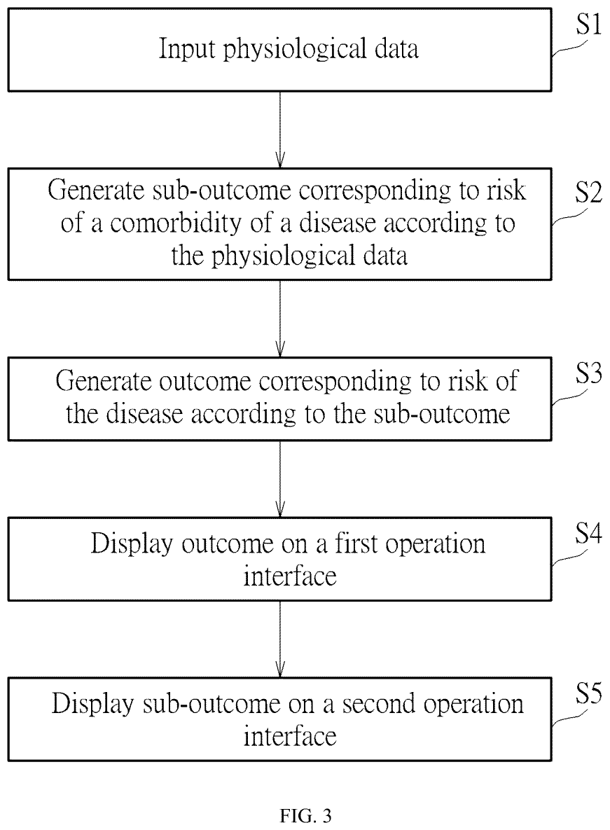

Furthermore, the operational relationships between the elements of the system 1 of the present disclosure as illustrated in FIG. 1 may be understood through a flow chart of steps involved in a method for predicting a disease risk of a subject as depicted in FIG. 3. In at least one embodiment, in the method of the present disclosure, at step S1, the input module 100 may be configured to input feature data of a female subject. In various embodiments of the present disclosure, the feature data may be qualitative or quantitative. In at least one embodiment of the present disclosure, some of the feature data may be binary. Also, in at least one embodiment of the present disclosure, some of the feature data may not have specific values associated therewith. In some embodiments of the present disclosure, one or more of the feature data may not be available in a subject. In some embodiments of the present disclosure, the missing feature data in a subject may be imputed from a median value of the feature data.

In at least one embodiment of the present disclosure, the feature data may include demographic data, information on cancer status, presence of comorbidity, treatment received, medication received, laboratory test data, or any combination thereof. In at least one embodiment of the present disclosure, the demographic data may include age, body mass index (BMI), smoking status, drinking status, and betel nut chewing status. In at least one embodiment of the present disclosure, the information on cancer status may include tumor size, cancer stage, expression of human epidermal growth factor receptor 2 (HER2), expression of estrogen receptor (ER), expression of progesterone receptor (PR), and a Ki-67 proliferation index. In at least one embodiment of the present disclosure, the presence of comorbidity may include a presence of hypertension, hyperlipidemia, renal disease, lung disease, diabetes, cerebrovascular disease, liver disease, congestive heart failure, dementia, rheumatic disease, and peptic ulcer disease. In some embodiments of the present disclosure, the presence of comorbidity may include a Charlson comorbidity index score. In at least one embodiment of the present disclosure, the treatment received may be a status of whether a surgery or a radiotherapy received. In at least one embodiment of the present disclosure, the medication received may include the status on receiving biguanide drugs, 3-hydroxy-3-methylglutaryl-coenzyme A (HMG-COA) reductase inhibitors, platelet aggregation inhibitors excluding heparin, beta-blocking agents, dihydropyridine derivatives, angiotensin II receptor blockers (ARBs), benzodiazepine derivatives, dipeptidyl peptidase-4 (DPP-4) inhibitors, sulfonylureas, or cyclooxygenase-2 (COX-2) inhibitors (coxibs). In at least one embodiment of the present disclosure, the laboratory test data may include albumin levels, creatinine levels, bilirubin levels, aspartate aminotransferase (AST) levels, alanine transaminase (ALT) levels, fasting glucose levels, white blood cell counts, red blood cell counts, and platelet counts. In at least one embodiment, the feature data of the present disclosure may include, but is not limited to, hormone evaluation results, blood pressure measurements, blood glucose (glycemia) measurements, blood lipid measurements, cholesterol measurements, age, gender, medical images, medical records, health survey records, physiological feature records, physiological function records, disease type records, medication records, living environment survey records, demographic information, and the like. In some embodiments of the present disclosure, the feature data may be input in the form of a set of values, ranges of values, cut-off values, and/or other suitable data storage formats.

In at least one embodiment of the present disclosure, at step S2, the sub-outcome analysis module 200, operatively coupled to the input module 100, may be configured to generate a sub-outcome indicative of a risk of a comorbidity associated with a disease based on the feature data. In some embodiments of the present disclosure, the sub-outcome analysis module 200 may utilize any suitable algorithm and/or statistical analysis method to determine a risk value of the comorbidity in a subject, using the feature data as input. In some embodiments of the present disclosure, the sub-outcome may include a risk value associated with one or more of the following: bone mass density conditions, such as a normal condition, a predicted deterioration, or a predicted improvement in bone mass density; osteoporosis; hypertension; hyperlipidemia; hyperglycemia; ovarian cancer; endometrial cancer; cervical cancer; colorectal cancer; breast cancer; and a drug response or side effect related to ongoing hormone replacement therapy.

In at least one embodiment of the present disclosure, the algorithm and/or statistical analysis method utilized by the sub-outcome analysis module 200 may be implemented as an artificial intelligence model trained or established using data retrieved from one or more of the following populations: the general female population, premenopausal females, perimenopausal females, menopausal females, or a postmenopausal females, regarding the relationship between a disease and a comorbidity of the disease. In some embodiments of the present disclosure, the artificial intelligence model may be implemented based on an algorithm. In at least one embodiment of the present disclosure, the algorithm may be a logistic regression, a linear discriminant analysis, a light gradient boosting machine, a gradient boosting machine, an extreme gradient boosting machine, an adaptive boosting machine (AdaBoost), a support vector classifier, a voting ensemble, a random forest, an artificial neural network, or any combination thereof. In some embodiments of the present disclosure, the artificial intelligence model may be built using one or more of the following approaches: a case control study for studying correlation and odds ratio between the factors of feature data of a female and a particular application field in predicting a disease risk of a female (as those illustrated in FIG. 2), a cohort study (e.g., a Kaplan-Meier curve method) for studying correlation between the factors of feature data with a comorbidity of a disease, a computation for hazard ratios (e.g., through a cox proportional hazards regression analysis) between the factors of feature data and a comorbidity of a disease, and a discussion of accuracy, sensitivity, and specificity of the artificial intelligence model in predicting a disease risk of a female. In some embodiments of the present disclosure, the risk value corresponding to the comorbidity, namely the sub-outcome, may be generated in a predetermined format (e.g., a value chart or a numerical report) in response to a display request by the subject or an operator conducting an examination on the subject.

In at least one embodiment of the present disclosure, at step S3, the outcome analysis module 300, operatively coupled to the sub-outcome analysis module 200, may be configured to generate an outcome corresponding to a risk of a disease based on a comprehensive assessment of the sub-outcomes. In some embodiments of the present disclosure, the outcome analysis module 300 may utilize the same or a different algorithm and/or statistical analysis method as the sub-outcome analysis module 200 to determine a comprehensive risk value of a disease, namely, the outcome of the subject, based on the risk values of the comorbidities associated with the disease. For example, the relationship between the comorbidities and the diseases may be referenced in Table 1 below.

TABLE 1
Relationship between comorbidities and diseases

| Disease | Comorbidity |
|---|---|
| Bone mass density- | Condition of bone mass density, osteoporosis, |
| related diseases | and drug response related to ongoing hormone |
| | replacement therapy |
| Metabolic syndromes | Hypertension, hyperlipidemia, hyperglycemia, |
| | and drug response related to ongoing hormone |
| | replacement therapy |
| Cancers | Ovarian cancer, endometrial cancer, cervical |
| | cancer, colorectal cancer, breast cancer, and |
| | drug response related to ongoing hormone |
| | replacement therapy |

Similarly, in at least one embodiment of the present disclosure, the risk value corresponding to the disease, namely the outcome, may indicate the current condition, predicted deterioration, and/or predicted improvement of the disease in the subject, and the outcome may be generated in a predetermined format (e.g., a value chart or a numerical report) in response to a display request from the subject or an operator conducting an examination on the subject.

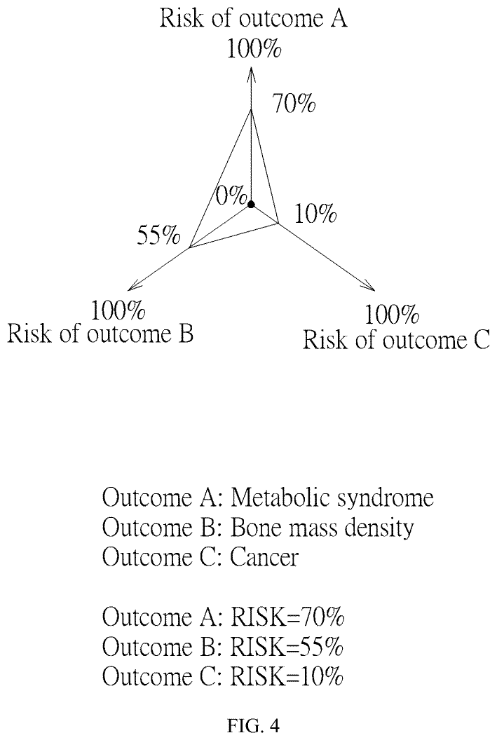

In at least one embodiment of the present disclosure, at step S4, the outcome display module 400, operatively coupled to the outcome analysis module 300, may be configured to present the outcome on a first operation interface of the outcome analysis module 300. In some embodiments of the present disclosure, the first operation interface may present a radar chart indicating the risk value of the disease in the subject. In at least one embodiment of the present disclosure, each axis on the radar chart may represent a risk value ranging from 0% to 100% of a corresponding disease, with the risk value of 0% sitting at a common origin of the radar chart and the risk value of 100% sitting at outskirts of the radar chart. In at least one embodiment of the present disclosure, as shown in FIG. 4, the radar chart on the first operation interface may consist of three axes, with an axis for Outcome A representing the risk value of metabolic syndromes, an axis for Outcome B representing the risk value of bone mass density-related diseases, and an axis for Outcome C representing the risk value of cancers. For example, Outcome A may indicate that the subject has a risk value of 70% in developing a metabolic syndrome, a risk value of 55% in developing a bone mass density-related disease, and a risk value of 10% in developing a cancer. Therefore, the radar chart presented by the first operation interface may intuitively indicate a treatment priority based on the risk values of the respective diseases for the subject.

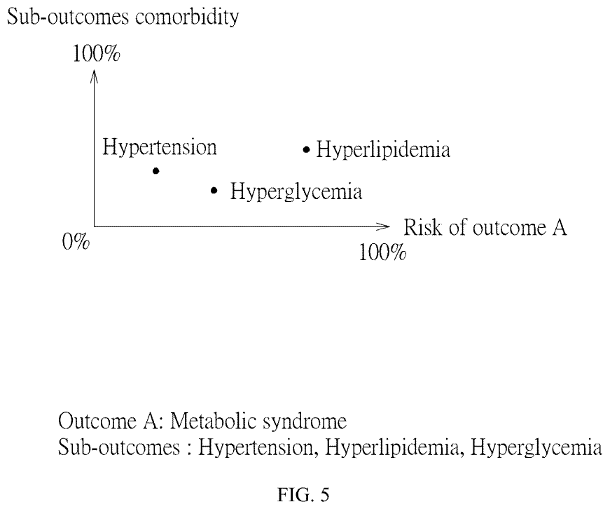

In at least one embodiment of the present disclosure, at step S5, the sub-outcome display module 500, operatively coupled to the sub-outcome analysis module 200 and the outcome display module 400, may be configured to display the sub-outcome on a second operation interface in response to an input selection made via the first operation interface presented by the outcome display module 400. In some embodiments of the present disclosure, as shown in FIG. 5, the second operation interface may present a two-dimensional scatter chart that indicates the risk value of a comorbidity associated with a specific disease in the subject. In at least one embodiment of the present disclosure, the X axis of the two-dimensional scatter chart may represent the risk value ranging from 0% to 100% of a selected disease of the subject, and the Y axis of the two-dimensional scatter chart may represent the risk value ranging from 0% to 100% of a comorbidity associated with the selected disease. The risk value of 0% is positioned at the common origin of the X axis and the Y axis, and each point on the two-dimensional scatter chart may indicate a comorbidity (sub-outcome) associated with the selected disease in the subject. For example, as shown in FIGS. 4 and 5, “Outcome A: Metabolic Syndrome” in FIG. 4 has a higher risk value than “Outcome B: Bone Mass Density” and “Outcome C: Cancer.” Accordingly, the subject may select “Outcome A: Metabolic Syndrome” on the first operation interface illustrated in FIG. 4 to view detailed examination results of Outcome A. In response, the second operation interface shown in FIG. 5 may be displayed by the sub-outcome display module 500 to reveal the comorbidities associated with metabolic syndrome, which may include hyperlipidemia (with the highest risk value), hypertension, and hyperglycemia (with the lowest risk value), respectively. Therefore, the two-dimensional scatter chart presented by the second operation interface may intuitively indicate a treatment priority for controlling the risk values of respective comorbidities associated with the selected disease in the subject.

In at least one embodiment of the present disclosure, a prediction model was built for 5-year survival of breast cancer patients in Taiwan, and a risk value of breast cancer was assessed using the system and method of the present disclosure. For example, a total of 44 data features were included, encompassing age; body mass index (BMI); smoking status; drinking status; betel nut chewing status; tumor size; cancer stage; expression status of human epidermal growth factor receptor 2 (HER2); expression status of estrogen receptor (ER); expression status of progesterone receptor (PR); Ki-67 proliferation index; presence of comorbidities including hypertension, hyperlipidemia, renal disease, lung disease, diabetes, cerebrovascular disease, liver disease, congestive heart failure, dementia, rheumatic disease, and peptic ulcer disease; Charlson comorbidity index score; treatment status regarding receipt of surgery and radiotherapy; medication status regarding receipt of biguanide drugs, 3-hydroxy-3-methylglutaryl-coenzyme A (HMG-COA) reductase inhibitors, platelet aggregation inhibitors excluding heparin, beta-blocking agents, dihydropyridine derivatives, angiotensin II receptor blockers (ARBs), benzodiazepine derivatives, dipeptidyl peptidase-4 (DPP-4) inhibitors, sulfonylureas, and cyclooxygenase-2 (COX-2) inhibitors (coxibs); and laboratory test data including albumin levels, creatinine levels, bilirubin levels, aspartate aminotransferase (AST) levels, alanine transaminase (ALT) levels, fasting glucose levels, white blood cell counts, red blood cell counts, and platelet counts. In at least one embodiment of the present disclosure, the prediction models were developed based on these input data features using 10 algorithms, including, but not limited thereto, logistic regression (LR), linear discriminant analysis (LDA), light gradient boosting machine (LGBM), gradient boosting machine (GBM), random forest (RF), adaptive boosting machine (AdaBoost), extreme gradient boosting machine (XGBoost), support vector classifier (SVC), voting ensemble, and artificial neural network (ANN).

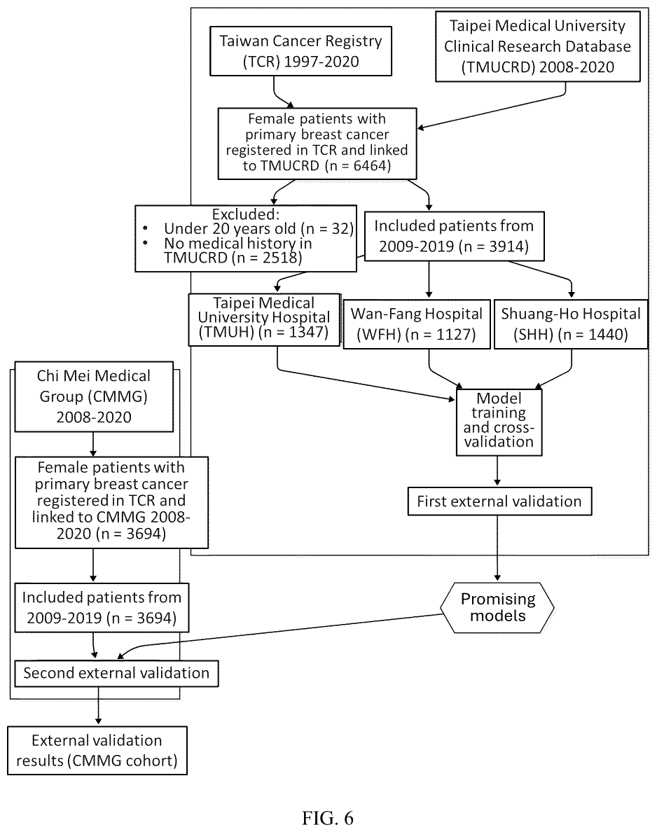

In at least one embodiment of the present disclosure, the study flow is shown in FIG. 6, where all female patients were diagnosed with primary breast cancer (International Classification of Disease for Oncology, Third Edition [ICD-O-3] code C50) from Jan. 1, 2009 to Dec. 31, 2019 in the Taiwan Cancer Registry (TCR) database. Subjects who were younger than 20 years of age and those without any medical history in all three hospitals of Taipei Medical University Hospital (TMUH), Wan-Fang Hospital (WFH), and Shuang-Ho Hospital (SHH) were excluded. A total of 3,914 subjects were included in this study.

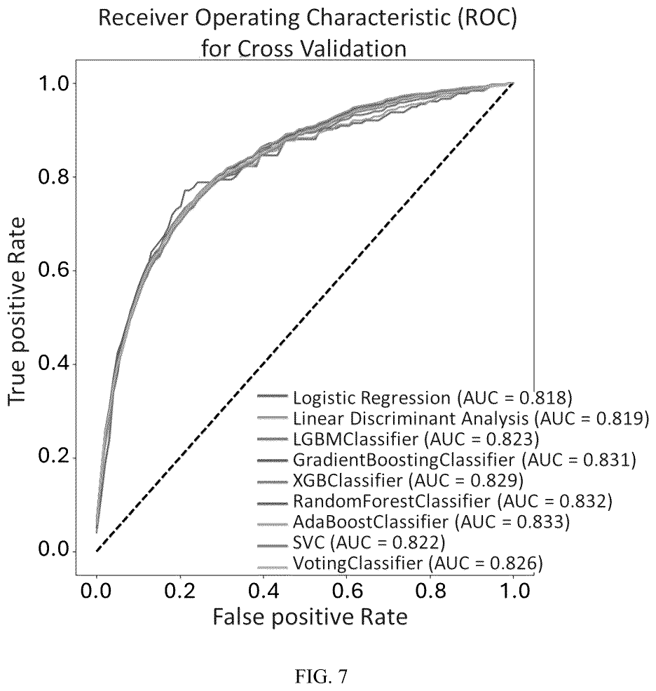

In order to build the prediction model, the training dataset included the patient data from TMUH and WFH. The data from TMUH and WFH were used for internal cross-validation, whereas the data from SHH were used for the first external validation. The validation results are summarized Table 2 and illustrated in FIG. 7. The results indicate that the models exhibited comparable areas under the receiver operating characteristic curve (AUCs) in the first external validation, with the model based on the random forest algorithm achieving the highest accuracy of 0.85.

TABLE 2
Prediction model for validation and evaluation

| | Cross | First | | | | | | |
|---|---|---|---|---|---|---|---|---|
| | validation | external | | | | | | |
| | AUC | validation | | | | | | |
| | (TMUH + | AUC | | | | | | F1- |
| Model | WFH) | (SHH) | Accuracy | Sensitivity | Specificity | NPV* | PPV** | score |
| Logistic | 0.82 | 0.79 | 0.72 | 0.75 | 0.72 | 0.96 | 0.23 | 0.35 |
| Regression | | | | | | | | |
| LDA | 0.82 | 0.79 | 0.79 | 0.71 | 0.80 | 0.96 | 0.29 | 0.41 |
| LGBM | 0.82 | 0.80 | 0.78 | 0.71 | 0.79 | 0.96 | 0.28 | 0.40 |
| GBM | 0.83 | 0.81 | 0.75 | 0.76 | 0.75 | 0.96 | 0.26 | 0.38 |
| XGBoost | 0.83 | 0.81 | 0.78 | 0.71 | 0.79 | 0.96 | 0.28 | 0.40 |
| Random | 0.83 | 0.81 | 0.85 | 0.67 | 0.87 | 0.96 | 0.37 | 0.48 |
| forest | | | | | | | | |
| AdaBoost | 0.83 | 0.81 | 0.75 | 0.76 | 0.74 | 0.96 | 0.25 | 0.38 |
| SVC | 0.82 | 0.80 | 0.80 | 0.56 | 0.83 | 0.94 | 0.27 | 0.37 |
| Voting | 0.83 | 0.80 | 0.84 | 0.69 | 0.86 | 0.96 | 0.36 | 0.47 |
| ensemble | | | | | | | | |
| ANN | 0.97 | 0.81 | 0.76 | 0.75 | 0.76 | 0.96 | 0.26 | 0.39 |

LDA: linear discriminant analysis; LGBM: light gradient boosting machine; GBM: gradient boosting machine; XGBoost: extreme gradient boosting machine; AdaBoost: adaptive boosting machine; SVC: support vector classifier; ANN: artificial neural network.
AUC: area under the receiver operating characteristic curve.
TMUH: Taipei Medical University Hospital; WFH: Wan-Fang Hospital; SHH: Shuang-Ho Hospital.
NPV*: Negative Predictive Value; PPV**: Positive Predictive Value.

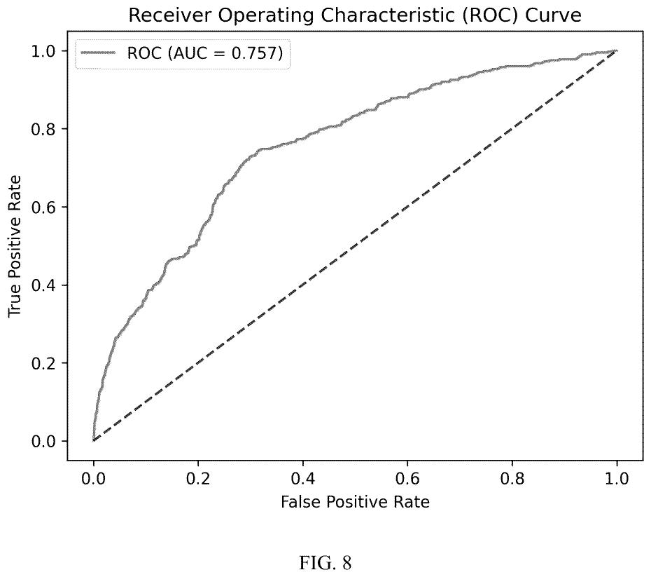

In addition, the data from Chi Mei Medical Group (CMMG) were used as the second external validation to evaluate the prediction model's performance. The validation and evaluation results are shown in Table 3 and FIG. 8.

TABLE 3
Prediction model evaluation results of second external validation

| Model | AUC | Accuracy | Sensitivity | Specificity | NPV* | PPV** | F1-score |
|---|---|---|---|---|---|---|---|
| Logistic | 0.68 | 0.78 | 0.45 | 0.82 | 0.92 | 0.24 | 0.31 |
| Regression | | | | | | | |
| LGBM | 0.74 | 0.68 | 0.68 | 0.68 | 0.95 | 0.21 | 0.32 |
| Random | 0.76 | 0.70 | 0.73 | 0.70 | 0.95 | 0.23 | 0.35 |
| Forest | | | | | | | |
| ANN | 0.67 | 0.72 | 0.53 | 0.75 | 0.93 | 0.20 | 0.29 |

LGBM: light gradient boosting machine; ANN: artificial neural network.
AUC: area under the receiver operating characteristic curve.
NPV*: Negative Predictive Value; PPV**: Positive Predictive Value.

Based on the above results, the present application provides a system and method of for predicting the disease risk of a female subject at least in the fields described below.

### I. For Metabolic Syndrome Examination/Cardiovascular Disease Examination

In at least one embodiment of the present disclosure, the analysis of the risk values for the outcome and the corresponding sub-outcome of metabolic syndromes may eliminate the need for long-term monitoring of a subject using multiple monitoring apparatuses simultaneously. Instead, the radar chart for the outcome and the two-dimensional scatter chart for the sub-outcome may be generated upon request once the feature data of the subject are input into the system 1 of the present disclosure. Further, the radar chart for the outcome and the scatter chart for the sub-outcome may indicate the risk of the subject developing a particular type of metabolic syndromes during a future period, as compared with that of the general female population, premenopausal females, perimenopausal females, menopausal females, and/or postmenopausal females. The future period may be 3 months, 6 months, 1 year, 2 years, 3 years, 4 years, 5 years, or any other desired time period from the time of examination. For example, if the system 1 of the present disclosure suggests that the subject is more likely to develop a metabolic syndrome due to hyperlipidemia rather than hyperglycemia within the next 3 to 6 months, the physician or clinician may prioritize treatment for hyperlipidemia to prevent the onset or development of the metabolic syndrome. In other words, the physician or clinician may prioritize an appropriate treatment for the subject based on a particular type of metabolic syndromes predicted and warned by the system 1 of the present stage at an early stage, thereby saving a substantial amount of medical resources, such as examination costs and medical manpower, without the need for prolonged monitoring typically required for a physician-based assessment that relies heavily on the physician's experience.

### II. For Bone Mass Density Examination

In at least one embodiment of the present disclosure, the analysis of the risk values for the outcome and the corresponding sub-outcome of the bone mass density-related diseases may help determine the development of osteoporosis in a female subject during a future period, as compared with that of the general female population, premenopausal females, perimenopausal females, menopausal females, and/or postmenopausal females. The future period may be 3 months, 6 months, 1 year, 2 years, 3 years, 4 years, 5 years, or any other desired time period from the time of examination. For example, if the system 1 of the present disclosure suggests that the bone mass density of the subject is currently at a T-score between 1 and 2.5 standard deviations below the average peak bone mass density in adult females (i.e., the low bone density, also referred to as osteopenia), and is estimated to decline to 2.5 standard deviations or more below the average peak bone mass density in adult females (i.e., osteoporosis) within two years, the physician or clinician may establish and plan personalized non-medical recommendations, health promotion programs, and/or medical treatments for the subject over the next two years to prevent further deterioration of osteoporosis. In other words, a physician or clinician may timely arrange a personalized treatment for osteoporosis according to the predicted loss of bone mass density identified by the system 1 of the present disclosure at an early stage.

### III. For Cancer Risk Examination

In at least one embodiment of the present disclosure, the analysis of the risk values for the outcome and the corresponding sub-outcome of cancer may assist in determining the potential development of cancer in a female subject during a future period, as compared with that of the general female population, premenopausal females, perimenopausal females, menopausal females, and/or postmenopausal females. The future period may be 3 months, 6 months, 1 year, 2 years, 3 years, 4 years, 5 years, or any other desired time period from the time of examination. For example, if the system 1 of the present disclosure suggests that the subject has a high risk value for developing breast cancer within one year, the physician or clinician may evaluate the necessity of taking actions such as surgery, radiation therapy, hormone replacement therapy, chemotherapy, or targeted drug therapy for the subject to prevent the development of breast cancer. In other words, the physician or clinician may timely prepare the female subject for cancer treatment based on the risk value of cancer determined by the system 1 of the present disclosure as early as upon completion of the examination.

### IV. For Treatment Response Prediction

In at least one embodiment of the present disclosure, the analysis of the risk values for the outcome and the corresponding sub-outcome of cancer may assist in determining potential results for treatment response in a female subject during a future period, as compared with those of the general female population, premenopausal females, perimenopausal females, menopausal females, and/or postmenopausal females. The future period may be 3 months, 6 months, 1 year, 2 years, 3 years, 4 years, 5 years, or any other desired time period from the time of examination. In at least one embodiment of the present disclosure, taking hormone replacement therapy as an example, if the system 1 of the present disclosure suggests that the effectiveness of ongoing hormone replacement therapy for the subject in treating a metabolic syndrome, a cardiovascular disease, a bone mass density-related disease, and/or a cancer may weaken within the next 3 to 6 months, the physician or clinician may evaluate adjustments to the medication method, drug type, drug dose, drug source, treatment schedule, the addition of non-pharmacological interventions (e.g., changes in lifestyle or dietary behavior), and/or clinical application for alternative therapies to improve treatment response. In other words, the physician or clinician may timely adapt an appropriate treatment for the subject based on the treatment effectiveness predicted by the system 1 of the present disclosure as soon as the examination is completed.

The present disclosure provides a system and a method for predicting the disease risk of a female subject, thereby enforcing female healthcare, including those going through menopause and at risk of developing female menopause-related diseases, aging-related diseases, cancers, and lifestyle-related diseases. The system and the method of the present disclosure for predicting the disease risk of a female subject may also provide insight into potential underlying causes of future health issues in the female subject at an early stage. For example, changes in physiological functionality and the endocrine system are inevitable in females during the aging process, and early detection of related diseases, such as bone mass density-related diseases, metabolic syndromes, cardiovascular diseases, and cancers, is therefore highly important for improving female healthcare. Accordingly, the system and the method of the present disclosure for predicting the disease risk of a female subject may evaluate the health risk of the female subject across different layers of analysis results (e.g., the outcome for a specified disease and the sub-outcome of a comorbidity of the specified disease) and assist physicians and clinicians in making timely decisions for formulating treatment based on the relationships between these layers of the analysis results. In doing so, the system and the method of the present disclosure for predicting the disease risk of a female subject may save a substantial amount of medical resources, such as examination costs and medical manpower, without the need for prolonged monitoring typically required for physician-based assessments, and may provide objective recommendations while eliminating subjective, biased determinations that rely heavily on the physician's experience.

Those skilled in the art will readily observe that numerous modifications and alterations of the system and method may be made while retaining the teachings of the disclosure. Accordingly, the above disclosure should be construed as limited only by the metes and bounds of the appended claims.

What is claimed is:

1. A system for predicting a disease risk of a female subject in need thereof, comprising:
an input module configured to input feature data of the female subject in need thereof;
a sub-outcome analysis module coupled with the input module and configured to generate a sub-outcome corresponding to a risk of a comorbidity of a disease according to the feature data;
an outcome analysis module coupled with the sub-outcome analysis module and configured to generate an outcome corresponding to a risk of the disease according to a comprehensive assessment of the sub-outcome;
an outcome display module coupled with the outcome analysis module and configured to present the outcome on a first operation interface of the outcome analysis module; and
a sub-outcome display module coupled with the sub-outcome analysis module and the outcome display module and configured to present the sub-outcome on a second operation interface of the sub-outcome analysis module in response to an input selection made via the first operation interface.

2. The system of claim 1, wherein the feature data is at least one of a demographic data, a cancer status, presence of a comorbidity, treatment received, medication received, laboratory test data, and any combination thereof.

3. The system of claim 2, wherein the demographic data is age, body mass index, smoking status, drinking status, or betel nut chewing status.

4. The system of claim 2, wherein the cancer status is tumor size, cancer stage, expression of human epidermal growth factor receptor 2, expression of estrogen receptor, expression of progesterone receptor, or a Ki-67 proliferation index.

5. The system of claim 2, wherein the presence of a comorbidity is the presence of hypertension, hyperlipidemia, renal disease, lung disease, diabetes, cerebrovascular disease, liver disease, congestive heart failure, dementia, rheumatic disease, or peptic ulcer disease.

6. The system of claim 2, wherein the presence of a comorbidity is a Charlson comorbidity index score.

7. The system of claim 2, wherein the treatment is a surgery or a radiotherapy.

8. The system of claim 2, wherein the medication is a biguanide drug, a 3-hydroxy-3-methylglutaryl-coenzyme A reductase inhibitor, a platelet aggregation inhibitor excluding heparin, a beta-blocking agent, a dihydropyridine derivative, an angiotensin II receptor blocker, a benzodiazepine derivative, a dipeptidyl peptidase-4 inhibitor, a sulfonylurea, or a cyclooxygenase-2 inhibitor.

9. The system of claim 2, wherein the laboratory test data is an albumin level, a creatinine level, a bilirubin level, an aspartate aminotransferase level, an alanine transaminase level, a fasting glucose level, a white blood cell count, a red blood cell count, or a platelet count.

10. The system of claim 1, wherein the disease is a bone mass density-related disease, a metabolic syndrome, or a cancer.

11. The system of claim 1, wherein the comorbidity is a bone mass density condition, osteoporosis, hypertension, hyperlipidemia, hyperglycemia, ovarian cancer, endometrial cancer, cervical cancer, colorectal cancer, breast cancer, or a drug response related to ongoing hormone replacement therapy.

12. The system of claim 1, wherein the disease is a bone mass density-related disease, and the comorbidity is a bone mass density condition, osteoporosis, or a drug response related to ongoing hormone replacement therapy, and wherein the bone mass density condition is a normal condition, a predicted deterioration, or a predicted improvement.

13. The system of claim 1, wherein the disease is a metabolic syndrome, and the comorbidity is hypertension, hyperlipidemia, hyperglycemia, or a drug response related to ongoing hormone replacement therapy.

14. The system of claim 1, wherein the disease is a cancer, and the comorbidity is ovarian cancer, endometrial cancer, cervical cancer, colorectal cancer, breast cancer, or a drug response related to ongoing hormone replacement therapy.

15. The system of claim 1, wherein the sub-outcome analysis module is configured to generate, by an artificial intelligence model, the sub-outcome according to the feature data.

16. The system of claim 15, wherein the artificial intelligence model is configured to perform an algorithm including a logistic regression, a linear discriminant analysis, a light gradient boosting machine, a gradient boosting machine, an extreme gradient boosting machine, an adaptive boosting machine, a support vector classifier, a voting ensemble, a random forest, an artificial neural network, or any combination thereof.

17. The system of claim 1, wherein the outcome analysis module is configured to generate, by an artificial intelligence model, the outcome according to a comprehensive assessment of the sub-outcome.

18. The system of claim 17, wherein the artificial intelligence model is configured to perform an algorithm including a logistic regression, a linear discriminant analysis, a light gradient boosting machine, a gradient boosting machine, an extreme gradient boosting machine, an adaptive boosting machine, a support vector classifier, a voting ensemble, a random forest, an artificial neural network, or any combination thereof.

19. A method for predicting a disease risk of a female subject in need thereof, comprising:
collecting feature data of the female subject;
generating a sub-outcome corresponding to a risk of a comorbidity of a disease according to the feature data;
generating an outcome corresponding to a risk of the disease according to a comprehensive assessment of the sub-outcome;
displaying the outcome on a first operation interface of an outcome analysis module; and
displaying the sub-outcome on a second operation interface of a sub-outcome analysis module in response to an input selection made via the first operation interface.

20. The method of claim 19, wherein at least one of the sub-outcome and the outcome is generated by an artificial intelligence model configured to perform an algorithm selected from the group consisting of a logistic regression, a linear discriminant analysis, a light gradient boosting machine, a gradient boosting machine, an extreme gradient boosting machine, an adaptive boosting machine, a support vector classifier, a voting ensemble, a random forest, an artificial neural network, and any combination thereof.
